(19) United States
(12) Patent Application Publication
(10) Pub. No.: US 2019/0201511 A1
(43) Pub. Date: Jul. 4, 2019

(54) Placental Based Contraceptive Vaccine

(71) Applicant: BATU BIOLOGICS, INC., San Diego, CA (US)

(72) Inventors: Samuel C. Wagner, San Diego, CA (US); Thomas E. Ichim, San Diego, CA (US); Santosh Kesari, San Diego, CA (US)

(21) Appl. No.: 16/239,346

(22) Filed: Jan. 3, 2019

Related U.S. Application Data
(60) Provisional application No. 62/613,532, filed on Jan. 4, 2018.

(51) Int. Cl. A61K 39/00

(52) U.S. Cl. CPC A61K 39/0006; A61K 2039/572; A61K 2039/55; A61K 2039/515; A61K 2039/575

(57) ABSTRACT

Disclosed are methods of preventing pregnancy through induction of immunological responses through vaccination with placental extracts and products associated with placentation. In one particular embodiment an immune response is triggered towards pregnancy associated neoangiogenesis through administration of placental endothelial cells capable of triggering immunity. In other embodiments the process of replicating immunological events associated with pregnancy failure are recapitulated by stimulation of immunity to antigens such as VEGFR-1, VEGFR-2, CD105, FGF1-R, Integrin αvβ3, and CD-248.

### CROSS-REFERENCE TO RELATED APPLICATIONS

This Patent Application takes priority from Provisional Patent Application No. 62/613,532, titled Placental Based Contraceptive Vaccine, filed on Jan. 4, 2018, the contents of which are expressly incorporated herein by this reference as though set forth in their entirety and to which priority is claimed.

### FIELD OF THE INVENTION

The invention pertains to the field of immunotherapy, specifically the invention pertains to the use of immunotherapy as a means of contraception, more specifically, the invention pertains leveraging of immune responses to the FDA Phase I cleared cancer vaccine ValloVax as a means of preventing pregnancy.

### BACKGROUND OF THE INVENTION

At present there are a variety of contraceptives and contraceptive devices that are available. However, each is accompanied by certain drawbacks. For example, diaphragms require careful fitting, usually by a trained physician, rendering them ill-suited for underdeveloped countries, where needed most. Further, condoms can tear, spent condoms must be disposed of and can feel unnatural.

While claims are made that the birth control pill is safe, evidence exists to the contrary. In particular, women over 35, who are heavy smokers (more than 14 cigarettes a day), are obese, or have (or have a history of) diabetes, high blood pressure, high cholesterol, cancer of the breast or sex organs, blood clots, heart attack or stroke have a significantly increased risk of serious side effects (including a heart attack or stroke) while taking the pill. This risk increases with age. Less severe side effects, including nausea and vomiting, breast tenderness and engorgement, acne, fluid retention, weight gain, increased vaginal discharge and breakthrough bleeding, can be experienced, particularly when a female first takes the pill. Spermicidal contraceptives, which typically contain surfactants, can negatively affect normal vaginal flora. For example, frequent use of N-9 as a vaginal contraceptive/microbiocide has been associated with an increased risk of vaginal or cervical infection, irritation, or ulceration.

Additionally, methods of contraception include surgical sterilization of women and oral contraceptive use by women, which are the most common methods of contraception in the U.S. Hormone modulating contraceptives include combined (estrogen/progestin) contraceptives, such as combined injectable contraceptives, combined oral contraceptives; and progestin-only contraceptives, such as norplant implants, progestin-only injectable contraceptives, or progestin-only pills. However, combined estrogen/progestin oral contraceptives may cause or lead to thromboembolic disorders, cerebrovascular accidents, coronary artery disease, liver abnormalities, estrogen dependent cancers, and pregnancy. Other methods of contraception include intrauterine devices; cervical cap; barrier methods, such as male condoms, female condoms, diaphragms, spermicides, or contraceptive sponge; and rhythm methods, which are highly dependent on the individuals involved. Many of these methods may have far reaching side effects both physiologically and physically. Thus, there is a need for improved methods of regulating fertility as well as methods for diagnosing fertility for both humans and a variety of domestic and wild animals.

To address the above mentioned shortcomings, several investigators have utilized vaccine based approaches for contraception, these include the hCG vaccine, the GnRF vaccine, (Improvest) and testosterone. Unfortunately none of these approaches are approved for human use, and efficacy has been limited, in part due to their monovalent properties.

### DESCRIPTION OF THE INVENTION

The invention provides immunologically based contraceptive methods through stimulation of antibody and cellular response targeting pregnancy associated angiogenesis. In one embodiment of the invention the clinical stage cancer vaccine ValloVax, described in the following references, is utilized as a stimulator of immunity capable of producing a contraceptive effect in immune competent females. Other preparations of placental antigenic compositions may be used including xenogeneic, or other allogeneic preparations capable of triggering immunity to cancer angiogenesis.

The basis of the anticontraceptive effect resides in ability of placental preparations to induce immunity towards various antigens associated with pregnancy, as well as hormones, peptides and factors necessary for initiation of pregnancy. One such example is, chorionic gonadotropin (CG), e.g. human chorionic gonadotropin (hCG), is secreted by cells of the human placenta and blastocyst, but not by the unfertilized ovum. The immunogenicity of peptides associated with pregnancy

Additional references describe the active production of anti-hCG antibodies in tumor-bearing animals following stimulation by an hCG vaccine. (See, e.g., U.S. Pat. Nos. 5,762,931; and 4,780,312.)

Direct vaccination with intact, protein combinations containing placentally derived proteins, including, chorionic gonadotropin antigens is disclosed with the purpose of vaccinating so as to enter into the Class II MHC pathway of antigen presentation and to result in a CD4+ helper T cell-mediated immune response. Additionally, the invention teaches means of promoting cross presentation, using means to promote dendritic cell uptake so as to stimulate CD8+ cytotoxic T cell-mediated cellular immune response. In some embodiments of the invention, exoosmes from placenta are utilized in a manner similarly described in the case of muscle exosomes, in order to promote cross presentation and CD8 cytotoxic cells. In other embodiments, agents such as GM-CSF are administered together with ValloVax or other placental preparations in order to promote cross-presentation and thus increase CD8 cytotoxic T cell responses. To enhance cross presentation, GM-CSF may be administered together with various toll like receptor agonists such as CpG DNA, or with interferon alpha.

The present invention further provides methods for fertility control based on inducing an immune response to pregnancy associated proteins, including chorionic gonadotropin (hCG), or a subunit thereof. The present invention addresses one or more of the drawbacks inherent in the prior art by providing novel methods for generating a multivalent immune response against immunogenic epitopes of an endogenous protein, hCG, in vivo in a subject, as an effective method of immunotherapy.

In one embodiment, the invention relates to hCG-containing placental extracts, placental endothelial cells, trophoblasts, and exosomes thereof. The invention teaches that such sources of proteins can be used as a means for stimulating immune responses adverse to fertility.

The “immune response” conferred by the methods of the invention can be a humoral (antibody) and/or a cell mediated immune response to one or more immunogenic epitopes of placental preparations, but more importantly interferes with fertility.

In one aspect, administration of a placental vaccine of the present invention is effective to result in an antigen-specific cytotoxic T-lymphocytes (CTL)-mediated response against major histocompatibility complex class I (MHC-I) restricted peptides of proteins necessary for fertility. In another aspect, the vaccines of the present invention are effective to elicit a T cell mediated humoral immune response resulting in production of antibodies by the subject which are directed against one or more pregnancy protein associated immunogenic epitopes. In a further aspect the vaccines of the present invention are effective to elicit both a T helper 1 (Tₕ₁) and T helper 2 (Tₕ₂) type of T cell response by the subject. The invention further provides methods of administering a placental vaccine to a subject comprising an adjuvant which stimulates antigen presentation in order to facilitate and effective immune response capable of inhibiting pregnancy or blocking fertilization. The desired placental contraceptive vaccine is a placental preparation containing immunogenic epitope(s) or a precursors of an immunogenic epitopes of proteins that include hCG, leutinizing hormone (LH) or follicle stimulating hormone (FSH).

In one aspect, the vaccines of the present invention includes placental preparations combined with immunogenic cytokines and/or added hCG immunogenic peptide together with the cytokine or immune system stimulator in the same host cell. In an additional aspect, the invention provides methods for eliciting an immune response to a number of immunogenic epitopes by administering to a subject, an expression vector comprising fragments of a library derived from the nucleotide sequence which encodes placental derived growth factor.

Unless defined differently, all technical and scientific terms used herein have the same meanings as commonly understood by one of skill in the art to which the disclosed invention belongs. In particular, the following terms and phrases have the following meaning.

The term “Angiogenesis” means any alteration of an existing vascular bed or the formation of new vasculature which benefits tissue perfusion. This includes the formation of new vessels by sprouting of endothelial cells from existing blood vessels or the remodeling of existing vessels to alter size, maturity, direction or flow properties to improve blood perfusion of tissues. As used herein the terms, “angiogenesis,” “revascularization,” “increased collateral circulation,” and “regeneration of blood vessels” are considered as synonymous.

The terms “antigenic precursor” or “precursor” relative to immunogenic epitopes, as used herein refer to peptides capable of being processed to immunogenic peptides by the cells of the subject.

The term “hCG peptide” and “hCG epitope” refer to an amino acid sequence which is the same as part of but not all of the amino acid sequence of the entire hCG protein, and which retains at least one biological function or activity of the entire hCG protein, for example, a fragment which retains an immunological activity of the full hCG protein.

The term “hCG immunogenic polypeptide” or “hCG immunogenic beta subunit polypeptide” or fragments thereof as used herein refer to amino acid sequences derived from hCG or the beta subunit of hCG, respectively, which are capable of eliciting a cellular and/or humoral immune response when exposed to the immune response cells of an immunocompetent subject. Such an immune response may require antigen processing in conjunction with class I and/or H major histocompatability antigens (MHC).

The terms “Class II major histocompatability complex”, “Class II MHC” and “Class II”, as used herein refer to molecules that are expressed on various cell types and which play an essential role in the recognition of protein antigens by T cells. Class II MHC molecules typically bind peptides of from about 7 to 30 or more amino acids and form complexes that are recognized by antigen-specific CD4+ T cells. Such peptide/CD4+ T cell complexes facilitate antibody production against the peptide antigen by an immunocompetent subject.

The term “immune response” as used herein refers to a cellular immune response such as a cytotoxic T cell response and/or a humoral immune response such as production of antibodies against an immunogenic epitope.

The term “immunocompetent subject”, as used herein refers to a subject having immune response cells which upon exposure to an immunogenic epitope, is capable of mounting a cellular and/or humoral immune response against the immunogenic epitope. The invention is useful for both the human and other mammalian subjects.

The term immunogenic “epitope” or “antigenic determinant”, as used herein refers to a portion of the amino acid sequence which will generate a T- and/or B-cell mediated immune response. It is preferred that the epitope be unique; that is, an immune response generated to the specific epitope show little or no cross-reactivity with other antigens.

The term “active immunization”, as used herein means the administration of a vaccine which induces an immune response by the immune response cells of the subject. “Active immunization” may be achieved by exposure of the immune response cells of the subject to a nucleic acid sequence or an amino acid sequence.

The term “exposing”, as used herein means bringing the immune response cells of the subject in contact with a nucleic acid construct. Such “exposing”, may take place in vitro, e.g., by introduction of the construct into a host cell by calcium phosphate transfection, DEAE-Dextran mediated transfection, or electroporation, (Davis, L., Dibner, M., and Battey, I. BASIC METHODS IN MOLECULAR BIOLOGY, 1986), or in vivo, e.g., by introduction of a “naked” nucleic acid construct into a host by injection into muscle or other tissue (Wolf et al., 1990).

The term “passive immunization”, as used herein is meant the direct administration of antibodies to a subject, as an immunization approach.

The term “immune response cells”, as used herein refers to the cells of a subject which are capable of processing antigens and presenting them in conjunction with Class I or Class II MHC.

The term “polynucleotide” as used herein refers to a polymeric molecule having a backbone which supports bases capable of hydrogen bonding to typical polynucleotides, where the polymer backbone presents the bases linked by phosphodiester bonds in a manner to permit such hydrogen bonding in a sequence specific fashion between the polymeric molecule and a typical polynucleotide (e.g., single-stranded DNA). “Polynucleotides” include polymers having modifications, e.g., those involving phosphodiamidate morpholine (PMO) chemistry.

The term “recombinant nucleic acid”, as used herein refers to a nucleic acid sequence originally formed in vitro, generally by the manipulation of the nucleic acid by endonucleases, in a form not normally found in nature.

The term “homology” or “homologue” as used herein refers to the level of identity between two sequences, i.e., 70% homology means the same thing as 70% sequence identity when determined by the algorithms described below, and accordingly a homologue of a given sequence has at least about 70% or 80%, preferably about 80%, 85%, 90% or 95% sequence identity over a given length of the sequence.

Two nucleic acid fragments are considered to be “selectively hybridizable” to a reference polynucleotide if they are capable of specifically hybridizing to the polynucleotide or variants thereof or of specifically priming a polymerase chain amplification reaction: (i) under moderate or high stringency hybridization and wash conditions, as described, for example, in Maniatis, et al. (1982), pages 320-328, and 382-389; (ii) under reduced stringency wash conditions that allow at most about 25-30% base pair mismatches, for example: 2.times.SSC (contains sodium 3.0 M NaCl and 0.3 M sodium citrate, at pH 7.0), 0.1% sodium dodecyl sulfate (SDS) solution, room temperature twice, 30 minutes each; then 2.times.SSC, 0.1% SDS, 37.degree. C., once, for 30 minutes; then 2.times.SSC, at room temperature twice, for 10 minutes each; or (iii) under standard PCR priming and amplification conditions (for example, as described in Saiki, et al., 1988), which result in specific amplification of sequences of the desired target sequence or its variants.

Generation of placental based vaccines useful for the practice of the current invention includes derivation of endothelial cells extracted from placental tissue, isolated into a homogeneous or semi-homogeneous mixture, treated with agents capable of augmenting immunogenicity, and subsequently administered into a recipient in which immune response to proliferating endothelium is desired. In one specific example, endothelial cells are purified from a human placenta according to the following steps: a) Fetal membranes are manually peeled back and the villous tissue is isolated from the placental structure, with caution being used not to extract the deciduas or fibrous elements of the placental structure; b) The fetal villous tissue is subsequently washed with cold saline to remove blood and scissors are used to mechanically digest the tissue into pieces as small as possible; c) The minced tissue is then enzymatically digested. Specifically, about 25 grams of minced tissue is incubated with approximately 56 ml of liquid solution which has been pre-warmed to a temperature of 37 Celsius. Said solution comprised of Hanks Buffered Saline Solution (HBSS) supplemented with 25 mM of HEPES and containing Calcium and Magnesium, said solution containing 0.28% collagenase, 0.25% dispase, and 0.01% DNAse (added during the incubation periods as described below); d) The mixture of minced placental villus tissue and digesting solution is incubated under stirring conditions for three incubation periods of 20 minutes each. Ten minutes after the first incubation period and immediately after the second and third incubation periods, the DNAse is added to make up a total concentration of DNase, by volume, of 0.01%; e) In the first and second incubations, the incubation flask is set at an angle, and the tissue fragments are allowed to settle for approximately 1 minute, with 35 ml of the supernantant cell suspension being collected and replaced by 38 ml (after the first digestion) or 28 ml (after the second digestion) of fresh digestion solution. After the third digestion the whole supernatant is collected; f) The supernatant collected from all three incubations is pooled and is poured through approximately four layers of sterile gauze and through one layer of 70 micro meter polyester mesh. The filtered solution is then centrifuged for 1000 g for 10 minutes through diluted new born calf serum, said new born calf serum diluted at a ratio of 1 volume saline to 7 volumes of new born calf serum; g) The pooled pellet is then resuspended in 35 ml of warm DMEM with 25 mM HEPES containing 5 mg DNase I; h) The suspension is then mixed with 10 ml of 90% Percoll to give a final density of 1.027 g/ml and is centrifuged at 550 g for 10 minutes with the centrifuge brake off; i) The pellet is then collected and resuspended in 15 ml of DMEM with 25 mM HEPES that is layered over a discontinuous Percoll gradient comprising of 20%-70% Percoll in 10% steps and centrifuged at 1900 g for 20 minutes; j) The cells found at the 1.037 g/ml and 1.048 g/ml are collected utilized for the generation of a cellular vaccine product.

Said cellular vaccine product from step “j”, in a preferred embodiment is treated with an agent capable of augmenting immunogenicity. Said immunogenicity in this context refers to ability to enhance recognition by recipient immune system. In one embodiment, immunogenicity refers to enhanced expression of HLA I and/or HLA II molecules. In another embodiment, immunogenicity refers to enhanced expression of costimulatory molecules. Said costimulatory molecules are selected from a group comprising of: CD27; CD80; CD86; ICOS; OX-4; and 4-1 BB. In another embodiment, immunogenicity refers to enhanced ability to stimulate proliferation of allogeneic lymphocytes in a mixed lymphocyte reaction. Immunogenicity may be augmented by incubation with one of the lymphokine or cytokine proteins that are known in the art, or with a member of the interferon family. In one particular embodiment, said purified endothelial cells are incubated with interferon gamma. In one particular embodiment, interferon gamma is incubated with endothelial cells, whether purified or unpurified for a period of approximately 48 hours, at a concentration of approximately 150 IU/ml.

Endothelial cells may be expanded after purification as described above before treatment with agents capable of augmenting immunogenicity. For example, endothelial cells may be treated with an endothelial cell mitogen. Said endothelial cell mitogen may be any protein, polypeptide, variant or portion thereof that is capable of, directly or indirectly, inducing endothelial cell growth. Such proteins include, for example, acidic and basic fibroblast growth factors (aFGF) (GenBank Accession No. NP₋₁₄₉₁₂₇) and bFGF (GenBank Accession No. AAA52448), vascular endothelial growth factor (VEGF) (GenBank Accession No. AAA35789 or NP₋₀₀₁₀₂₀₅₃₉), epidermal growth factor (EGF) (GenBank Accession No. NP₋₀₀₁₉₅₄), transforming growth factor α (TG-Fα) (GenBank Accession No. NP₋₀₀₃₂₂₇) and transforming growth factor β (TFG-β) (GenBank Accession No. 1109243A), platelet-derived endothelial cell growth factor (PD-ECGF) (GenBank Accession No. NP₋₀₀₁₉₄₄), platelet-derived growth factor (PDGF) (GenBank Accession No. 1109245A), tumor necrosis factor α (TNF-α) (GenBank Accession No. CAA26669), hepatocyte growth factor (HGF) (GenBank Accession No. BAA14348), insulin like growth factor (IGF) (GenBank Accession No. P08833), erythropoietin (GenBank Accession No. P01588), colony stimulating factor (CSF), macrophage-CSF (M-CSF) (GenBank Accession No. AAB59527), granulocyte/macrophage CSF (GM-CSF) (GenBank Accession No. NP₋₀₀₀₇₄₉), monocyte chemotactic protein-1 (GenBank Accession No. P13500) and nitric oxide synthase (NOS) (GenBank Accession No. AAA36365). See, Klagsbrun, et al., Annu. Rev. Physiol., 53:217-239 (1991); Folkman, et al., J. Biol. Chem., 267:10931-10934 (1992) and Symes, et al., Current Opinion in Lipidology, 5:305-312 (1994). Variants or fragments of a mitogen may be used as long as they induce or promote endothelial cell or endothelial progenitor cell growth. Preferably, the endothelial cell mitogen contains a secretory signal sequence that facilitates secretion of the protein. Proteins having native signal sequences, e.g., VEGF, are preferred. Proteins that do not have native signal sequences, e.g., bFGF, can be modified to contain such sequences using routine genetic manipulation techniques. See, Nabel et al., Nature, 362:844 (1993). Before expansion, endothelial cells may be further purified based on expression of surface receptors using affinity-based methodologies that are known to one of skill in the art, said methodologies include magnetic activated cell sorting (MACS), cell panning, or affinity chromatography. Other methodologies such as fluorescent activated cell sorting (FACS) may also be used. Various lectins are known to have selectivity to endothelial cells, for example, Ulex europaeus agglutinin I is known to possess ability to bind to endothelial cells and endothelial progenitor cells. It is within the scope of the current invention to define “endothelial cell” as including “endothelial progenitor cell”.

The contraceptive vaccine formulation may be utilized in conjunction with known adjuvants in order to induce an immune response that is Th1 or Th17-like, and which will inhibit the proliferation of endothelial cells in the recipient. Such adjuvant compounds are known in the art to boost the activity of the immune system and are now under study as possible adjuvants, particularly for vaccine therapies. Some of the most commonly studied adjuvants are listed below, but many more are under development. For example, Levamisole, a drug originally used against parasitic infections, has recently been found to improve survival rates among people with colorectal cancer when used together with some chemotherapy drugs. It is often used as an immunotherapy adjuvant because it can activate T lymphocytes. Additionally, the compound has been demonstrated to induce maturation of dendritic cells, further supporting an immune modulatory role. Levamisole is now used routinely for people with some stages of colorectal cancer and is being tested in clinical trials as a treatment for other types of cancer. Additionally, it has been shown to augment efficacy of other immunotherapeutic agents such as interferon. Aluminum hydroxide (alum) is one of the most common adjuvants used in clinical trials for cancer vaccines. It is already used in vaccines against several infectious agents, including the hepatitis B virus. Bacille Calmette-Guerin (BCG) is a bacterium that is related to the bacterium that causes tuberculosis. The effect of BCG infection on the immune system makes this bacterium useful as a form of anticancer immunotherapy. BCG was one of the earliest immunotherapies used against cancer, either alone, or in combination with other therapies such as hormonal, chemotherapy or radiotherapy. It is FDA approved as a routine treatment for superficial bladder cancer. Its usefulness in other cancers as a nonspecific adjuvant is also being tested or has demonstrated therapeutic effects. Researchers are looking at injecting BCG to give an added stimuli to the immune system when using chemotherapy, radiation therapy, or other types of immunotherapy.

Thus in various embodiments of the current invention, one of skill in the art is directed towards references which have utilized BCG as an adjuvant for other therapies for concentrations and dosing regimens that would apply to the current invention for elicitation of immunity towards proliferating endothelial cells. Incomplete Freund's Adjuvant (IFA) is given together with some experimental therapies to help stimulate the immune system and to increase the immune response to cancer vaccines, both protein and peptide in part by providing a localization factor for T cells. IFA is a liquid consisting of an emulsifier in white mineral oil. Another vaccine adjuvant useful for the present invention is interferon alpha, which has been demonstrated to augment NK cell activity, as well as to promote T cell activation and survival. QS-21 is a relatively new immune stimulant made from a plant extract that increases the immune response to vaccines used against melanoma. DETOX is another relatively new adjuvant. It is made from parts of the cell walls of bacteria and a kind of fat. It is used with various immunotherapies to stimulate the immune system. Keyhole limpet hemocyanin (KLH) is another adjuvant used to boost the effectiveness of cancer vaccine therapies. It is extracted from a type of sea mollusc. Dinitrophenyl (DNP) is a hapten/small molecule that can attach to tumor antigens and cause an enhanced immune response. It is used to modify tumor cells in certain cancer vaccines.

In one embodiment of the invention proliferating endothelial cells treated with an agent to stimulate immunogenicity are lysed and protein extracts are extracted and utilized as a vaccine. In some embodiments specific immunogenic peptides may be isolated for said cell lysate. In other embodiments, lyophilization of endothelial cells is performed subsequent to treatment with an agent that augments immunogenicity. In embodiments utilizing cellular extracts, various formulations may be generated. The formulations may conveniently be presented in unit dosage form and may be prepared by any of the methods well known in the art of pharmacy. Such methods include the step of bringing into association the active ingredient (for an antigenic molecule, construct or chimaeric polypeptide of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

In another embodiment of the invention placental immunization is performed in order induce an immune response which causes conditions unfavorable to pregnancy onset. For example, it is known that multiple immunological factors are unfavorable to pregnancy and these factors are usually similarly unfavorable to cancer. For example, both cancer and successful pregnancy are associated with activity of the immunogenic enzyme indolamine 2,3 deoxygenase (IDO). Ban et al examined women undergoing legal pregnancy termination and women with recurrent spontaneous abortion. Immunohistochemistry and real-time reverse transcription-polymerase chain reaction were used to analyze levels of IDO protein and mRNA in placenta, decidua and HTR-8/SVneo cells. Culture medium collected from trophoblast villous explant or HTR-8/SVneo cell cultures was used to measure IDO activity in response to interferon (IFN)-γ treatment. A total of 40 healthy women and 26 women with RSA provided samples of placenta and decidua. For normal pregnancies, IDO protein and mRNA was identified in placental trophoblasts, invasive extravillous trophoblasts and decidual glandular epithelium. IFN-γ significantly increased IDO activity in trophoblast villous explants and HTR-8/SVneo cells. Levels of IDO protein and mRNA in the placenta and decidua from normal pregnancies were significantly higher than in those from RSA. Therefore the authors concluded that decreased levels of IDO protein and mRNA in the placenta and decidua from RSA suggest an important role for IDO in the maintenance of normal pregnancy.

Zong et al performed another study in which they identified a novel function of IDO in regulating trophoblast cell proliferation and migration. They showed that IDO expression and activity were decreased in unexplained recurrent spontaneous abortion (URSA) compared to normal pregnancy. Furthermore, blocking IDO in human trophoblast cells led to reduced proliferation and migration, along with decreased STAT3 phosphorylation and MMP9 expression. Increased STAT3 phosphorylation reversed the IDO knockdown-suppressed trophoblast cell proliferation and migration. In addition, the overexpression of IDO promoted cell proliferation and migration, which could be abolished by the STAT3 signaling inhibitor (AG490). Finally, they observed similar reductions of STAT3 phosphorylation and MMP9 expression in URSA patients. These results indicate that the level of IDO expression may be associated with pregnancy-related complications, such as URSA, by affecting trophoblast cell proliferation and migration via the STAT3 signaling pathway.

Accordingly, in one embodiment of the invention placental immunization is utilized to trigger inhibitors of IDO, which resulted in decreased potential for pregnancy.

In one embodiment the invention provides a means of generating a population of cells with ability to inhibit endothelial cell proliferation. In one embodiment approximately 50 ml of peripheral blood is extracted from a patient in which inhibition of fertility is desired and peripheral blood monoclear cells (PBMC) are isolated using the Ficoll Method. PBMC are subsequently resuspended in approximately 10 ml RPMI media with 10% fetal calf serum and allowed to adhere onto a plastic surface for 2-4 hours. The adherent cells are then cultured at 37° C. in RPMI media supplemented with 1,000 U/mL granulocyte-monocyte colony-stimulating factor and 500 U/mL IL-4. This procedure, or a procedure similar to it, can be utilized for the generation of dendritic cells. Half of the volume of the GM-CSF and IL-4 supplemented media is changed every other day. Immature DCs are harvested on day 7. In one embodiment said generated DC are treated with endothelial cell extracts isolated from placental or otherwise proliferating endothelial cells. Said extracts are added to said immature dendritic cells on day 7. Endothelial pulsed dendritic cells may be administered directly as a vaccine, or may be utilized to stimulate autologous patient T cell clones in vitro. Said T cell clones may be selected for specificity to proliferating endothelial cells. Additionally, in some embodiments, whether for in vitro stimulation of T cells, or for direct use as a tumor vaccine, the endothelial cell pulsed dendritic cells may be further purified from culture through use of flow cytometry sorting or magnetic activated cell sorting (MACS), or may be utilized as a semi-pure population. In one embodiment DC are exposed to agents capable of stimulating maturation in vitro subsequent to pulsing with endothelial cell extracts. Specific means of stimulating in vitro maturation include culturing DC or DC containing populations with a toll like receptor agonist. Another means of achieving DC maturation involves exposure of DC to TNF-alpha at a concentration of approximately 20 ng/mL.

In another embodiment, a mixture of endothelial cells together with immature dendritic cells is used as a combination cellular vaccine. In another embodiment, endothelial cells (live or extracts or fixed) are administered in combination with dendritic cells together with activated T cells and/or NK cells. In order to activate T cells and/or NK cells in vitro, cells are cultured in media containing approximately 1000 IU/ml of interferon gamma. Incubation with interferon gamma may be performed for the period of 1 hour to the period of 14 days. Preferably, incubation is performed for approximately 48 hours, after which T cells and/or NK cells may be further stimulated via the CD3 and CD28 receptors. One means of accomplishing this is by addition of antibodies capable of activating these receptors. In one embodiment approximately, 3 ug/ml of anti-CD3 antibody is added, together with approximately 2 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 400 u/mL IL-2. Media containing IL-2 and antibodies may be changed every two days for approximately 7-24 days. In one particular embodiment DC are included to said T cells and/or NK cells in order to endow cytotoxic activity towards tumor cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or intralymphatically.

In some embodiments, endothelial cells are increased in immunogenicity by culture with T cells that are autologous or allogeneic to the donor of said endothelial cells. Said T cells may be activated by their allogeneic interaction with said endothelial cells, or may be introduced into contact with endothelial cells in an already preactivated state. In order to preactive T cells, firstly lymphocytes are collected and separation into the T cell population and cell sub-population containing a T cell can be performed, for example, by fractionation of a mononuclear cell fraction by density gradient centrifugation, or a separation means using the surface marker of the T cell as an index of detection. Subsequently, isolation based on surface markers may be performed. Examples of the surface marker include CD2, CD3, CD8 and CD4, and separation methods depending on these surface markers are known to one of skill in the art. For example, the step can be performed by mixing a carrier such as beads or a culturing flask onto which an anti-CD8 antibody has been immobilized (cell panning), with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as marker of detection and, for example, CD4 MicroBeads, Dynabeads M-450 CD4 can also be used.

In some embodiments of the invention, T regulatory cells are depleted before initiation of the culture, with the idea of “derepressing” suppressive elements within the heterogeneous T cell population. Depletion of T regulatory cells may be performed by negative selection by removing cells that express makers such as neuropilin, CD25, CD4, CD105, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-gamma, IL-15, IL-7, IFN-alpha, IL-12, CD40L, and IL-27. From the viewpoint of enhancing cellular immunity, particularly suitably, IL-2, IFN-gamma, or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1 alpha, MIP1 beta, CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes or antigen presenting cells (dendritic cell) presenting a target peptide such as a peptide derived from an endothelial cell antigen. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay such as the tetramer assay, a proliferation assay, a cytolytic cell assay, or an in vivo delayed hypersensitivity test using a recombinant endothelial cell-associated antigen or an immunogenic fragment or an antigen-derived peptide. In a preferred embodiment, the antigen derived peptides are specifically associated with proliferating endothelial cells, such as endothelial cells found in proximity to the tumor.

Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant endothelial-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay (ELISPOT), cytokine flow cytometry, a direct cytotoxity assay, measurement of cytokine mRNA by a quantitative reverse transcriptase polymerase chain reaction, or an assay which is currently used for measuring a T cell response such as a limiting dilution method.

In some aspects of the invention, it will be important to overcome tolerance that already exists to proliferating self endothelial cells. Accordingly, on of skill in the art is directed towards the following description of tolerogenic processes, with the knowledge that manipulation and specific inhibition of these processes is useful in the practice of the current invention. The argument has been made that tolerance is controlled to some extent by immature dendritic cells presenting self antigen in absence of costimulation/presence of co-inhibitors, which leads to generation of Treg cells and anergic T cells. This was demonstrated in several systems, for example, in a classical experiment Mahnke et al targeted the antigen ovalbumin to immature dendritic cells by conjugation to anti-DEC205 antibodies. It was demonstrated that antigen-specific Treg were generated, which was dependent on presentation by immature dendritic cells. In vivo relevance of Treg generated by targeting antigen to steady state dendritic cells can be seen in studies where DEC-205 targeting of antigen prevented autoimmune diabetes in a transgenic model system via FoxP3 expressing Treg.

We have reported on a “tolerogenic vaccine” created by ex vivo generation of immature DC treated with a chemical IKK inhibitor, and pulsed with collagen II, that was able to prevent arthritis in a mouse model. Similar tolerogenic uses of immature DC have been reported in diverse conditions such as transplantation, anti-Factor VIII immunity, autoimmune myocarditis, experimental autoimmune mysthenia gravis, and collagen induced arthritis. The possibility that in pregnancy the placental structures may be generating immature DC to protect themselves from T cell attack and/or generate Treg was suggested in studies showing tumor secreted VEGF would arrest DC maturation in vitro. Mechanistically it was demonstrated that VEGF blocks NF-kB activity in DC, which is a critical maturation-inducing factor. Given that VEGF is a primary cytokine in angiogenesis, the possibility of inhibited DC maturation being a mechanism of immune escape is attractive. Angiogenesis seems to be associated with various cells of the myeloid lineage. The myeloid suppressor cell, which will be described below, has been demonstrated stimulate angiogenesis directly, and through production of MMP-9 and VEGF. Myeloid suppressor cells have been demonstrated to play a role in pregnancy and maintaining fetal maternal tolerance.

One prospective observational study randomly recruited 85 women who underwent IVF treatment from May 2016 to June 2016 where investigated, the levels of peripheral blood granulocytic MDSC (G-MDSC), monocytic MDSC (M-MDSC) and their relations to IVF treatment outcomes were analyzed. the circulating G-MDSC level was significantly increased in the clinical pregnant group when compared to that in the nonclinical pregnant group (p=0.014), while M-MDSC had no significant difference. The G-MDSC level was an independent predictive factor for clinical pregnancy with odds ratios 12.7 (95% CI: 1.53-105.4, p=0.018) when using multiple logistic regression analysis. A receiver operating characteristic analysis (area under curve=0.634) found the clinical pregnancy rate in women with G-MDSC>2.38% was higher than that in women below this level (96 versus 66.7%, p=0.004). The high G-MDSC level in the peripheral blood was associated with clinical pregnancy, with a sensitivity of 37.5%, specificity of 95.2%. Accordingly the investigators concluded that high circulating G-MDSC level was associated with elevated clinical pregnancy rate. Accordingly, in one embodiment the invention teaches use of placental vaccination to reduce myeloid suppressor cells at a sufficient means to prevent fertility.

The invention includes the use of pulsing or administering to antigen presenting cells lysates, mRNA or peptides derived from ValloVax. The antigen presenting cells used in this invention are made by culturing stem cells in an environment that guides the progenitors towards (or promotes outgrowth on the desired cell type. In some instances, differentiation is initiated in a non-specific manner by forming embryoid bodies or culturing with one or more non-specific differentiation factors. Embryoid bodies (EBs) can be made in suspension culture: undifferentiated hPS cells are harvested by brief collagenase digestion, dissociated into clusters or strips of cells, and passaged to non-adherent cell culture plates. The aggregates are fed every few days, and then harvested after a suitable period, typically 4-8 days. Specific recipes for making EB cells from hPS cells can be found in U.S. Pat. No. 6,602,711 (Thomson); WO 01/51616 (Geron Corp.); US 2003/0175954 A1 (Shamblott & Gearhart); and US 2003/0153082 A1 (Bhatia, Robarts Institute). Alternatively, fairly uniform populations of more mature cells can be generated on a solid substrate: US 2002/019046 A1 (Geron Corp.). Maturation of the phagocytic or dendritic cell precursor is achieved in a subsequent step: potentially withdrawing the IL-3, but maintaining the GM-CSF, and adding IL-4 (or IL-13) and a pro-inflammatory cytokine. Other factors that may be helpful at this stage are IL-1 beta, interferon gamma (IFN gamma), prostaglandins (such as PGE2), and transforming growth factor beta (TGF beta); along with TNF alpha and/or IL-6 (FIG. 2). A more mature population of dendritic cells should emerge, having some of the characteristics described earlier.

Another embodiment of the present invention provides for a method of producing the composition/vaccine of the present invention and a method of activating the dendritic cells subsequent to administration of ValloVax or derivatives thereof to said dendritic cells. The method comprises providing dendritic cells; culturing the dendritic cells; pulsing the dendritic cells with tumor lysate and at least one TLR ligand. In various embodiments, the dendritic cells may be pulsed with tumor lysate at a concentration of about 50-1000 ug/10⁶-10⁷ PBMDCs, which can be effective at activating PBMDCs in vitro. The dendritic cells may be ones as described above. In a particular embodiment, the dendritic cells may be bone-marrow derived dendritic cells.

1. A method for prevention of fertility in a female comprising the steps of:
a) selecting an immune competent subject;
b) immunizing said subject with a composition capable of stimulating immunity towards pregnancy associated angiogenesis antigens; and
c) optionally repeating said immunization until sufficient immunity is generated to prevent induction of pregnancy.

2. The method of claim 1, wherein said pregnancy associated angiogenesis comprises endometrial angiogenesis, and wherein said endometrial angiogenesis comprises at least one of VEGF-predominated angiogenesis, endometrial proliferation in preparation for implantation, and leukemia inhibitory factor induced proliferation of endometrial regenerative cells.

3. The method of claim 1, wherein said pregnancy associated angiogenesis is angiogenesis associated with the corpus luteum, and wherein said corpus luteum angiogenesis is associated with enhanced VEGF production.

4. The method of claim 1, wherein said pregnancy associated angiogenesis is associated with placental angiogenesis, and wherein said placental angiogenesis is associated with at least one of enhanced production of human chorionic gonadotropin and trophoblast invasion into maternal myometrium.

5. The method of claim 1, wherein said angiogenesis associated antigens are selected from a group comprising of: a) CD-248; b) Robo 1-8; c) VEGF-r1; d) VEGF-R2; e) FGF1-R; f) FGF2-R; g) endosialin; h) Integrin αvβ3; i) PDGF-BB receptor; j) EGF-R; and k) human chorionic gonadotropin receptor.

6. The method of claim 1, wherein said composition capable of stimulating immunity towards pregnancy associated angiogenesis antigens utilizes endothelial cells as an immunogen.

7. The method of claim 6, wherein said endothelial cells are derived from the placenta, wherein the placenta is at least one of a hemochorial placenta, a term placenta, and a pre-term placenta.

8. The method of claim 7, wherein said endothelial cells are derived from the chorionic portion of the placenta.

9. The method of claim 6, wherein said endothelial cells are generated from a pluripotent stem cell population, wherein said pluripotent stem cell population is selected from a group of cells comprising of: a) embryonic stem cells; b) inducible pluripotent stem cells; c) somatic cell nuclear transfer generated stem cells; and d) parthenogenic stem cells.

10. The method of claim 6, wherein said endothelial cells are generated from endothelial precursor cells, wherein said endothelial precursor cells are obtained from a population of cells selected from a group comprising of: a) peripheral blood mononuclear cells; b) adipose tissue derived stromal vascular fraction; c) umbilical cord blood; e) perivascular tissue obtained from the wharton's jelly; and f) perivascular tissue obtained from the omentum.

11. The method of claim 10, wherein said endothelial precursor cells possess expression of the marker kdr-1.

12. The method of claim 11, wherein said endothelial cells possess replicative capacity upon isolation.

13. The method of claim 11, wherein said replicative capacity is endowed by culture in a media containing mitogens, wherein said mitogen is selected from a group of mitogens comprising of:
a) VEGF; b) IGF-; c) FGF-1; d) FGF-2; e) TGF-alpha; f) FGF-5; g) PDGF; h) EGF; i) IL-13; j) IL-20; k) NGF; l) BDNF; and m) HGF.

14. The method of claim 1 wherein said immunization is performed with an adjuvant, wherein said adjuvant is capable of at least one of increasing generation of cytotoxic T cells capable of suppressing pregnancy onset and stimulating generation of antibodies which inhibit pregnancy.

15. The method of claim 1, wherein said stimulation of immunogenicity of said endothelial cells is achieved through culture with an agent capable of upregulating HLA expression.

16. The method of claim 1, wherein said stimulation of immunogenicity of said endothelial cells is achieved through culture with an agent capable of upregulating expression of costimulatory molecules.

17. The method of 1, wherein said induction of immunogenicity in said endothelial cells is achieved through treatment of said endothelial cells with an agent capable of inducing signaling through a toll like receptor.

18. The method of claim 17, wherein the toll like receptor is at least one of TLR-2, TLR-3, TLR-4, TLR-5, TLR-7, TLR-8, and TLR-9.

19. The method of 1, wherein said induction of immunogenicity in said endothelial cells is achieved through treatment of said endothelial cells with an agent capable of inducing signaling through a Pathogen Associated Molecular Pattern (PAMP) receptor.

20. The method of claim 19, wherein said PAMP receptor is selected from a group comprising of: a) MDA5; b) RIG-1; and c) NOD.
